(19) United States
(12) Patent Application Publication
(10) Pub. No.: US 2023/0105348 A1
(43) Pub. Date: Apr. 6, 2023

(54) SYSTEM FOR ADAPTIVE HOSPITAL DISCHARGE

(71) Applicant: Siemens Healthcare GmbH, Erlangen (DE)

(72) Inventors: Asmir Vodencarevic, Fürth (DE); Walter Schmid, Seefeld (DE)

(21) Appl. No.: 17/485,717

(22) Filed: Sep. 27, 2021

(51) Int. Cl. G16H 40/20; G16H 10/60; G16H 50/30; G16H 50/20

(52) U.S. Cl. CPC G16H 40/20; G16H 10/60; G16H 50/20; G16H 50/30

(57) ABSTRACT

Systems and methods are provided for reliable individual readmission risk prediction. Information about an admitted patient is acquired. A model inputs the patient information and outputs a time-varying readmission risk prediction. The time-varying readmission risk prediction is presented in a relation to the Length of Stay (LoS) and the costs (actual costs and cost coverage). A treatment and/or discharge plan is generated that is implemented when a threshold risk is met.

### FIELD

The present embodiments relate to medical data processing.

### BACKGROUND

Acute hospital admissions are common for patients suffering from various diseases and especially for those suffering from chronic diseases such as Heart Failure (HF). HF is a global epidemic that poses a large societal burden not only for the patients and their families but also an economic burden for the healthcare system. The most frequently identified drivers that impact the incremental cost-effectiveness ratio are treatment costs and utility. Unplanned hospital admissions may happen due to multiple reasons and are often related to an abrupt worsening of the patient’s vital signs and symptoms. For example, an abrupt worsening in HF patients typically includes dyspnea, fatigue and swelling of feet and legs and is referred to as Acute Decompensated Heart Failure (ADHF). During an acute patient hospitalization, for example due to the ADHF, one or more procedures such as laboratory tests are performed and one or several treatments are administered with the purpose of improving and stabilizing the health status of the patient. After administering the treating for a certain time (e.g., several days or weeks), a clinician might decide to discharge the patient after considering the latest relevant medical parameters, for example, laboratory values and their trends (e.g., stabile normal blood pressure and oxygen saturation).

Decisions about discharging patients are typically made based on the best clinical judgements of the treating clinicians. Nevertheless, many patients are readmitted due to the same or similar reason oftentimes within relatively short timeframe after the patients have been discharged. Readmission after discharge is associated with clinical and financial burden to patients, hospitals, and society. As an example of a financial burden, readmission is used as a publicly reported metric with reimbursement implications to hospitals by the Centers for Medicare and Medicaid Services (CMS). For hospitals with excess risk-standardized readmission rates, CMS has lowered the reimbursement for certain clinical conditions such as acute myocardial infarction (AMI), heart failure (HF), pneumonia, chronic obstructive pulmonary disease, total hip arthroplasty, and total knee arthroplasty.

CMS and other entities have used their reimbursement policies to encourage hospitals to implement measures for avoiding readmissions. For example, to try to reduce avoidable readmissions, hospitals might impose policies to improve communication and care coordination, e.g., by extending the patients’ length of stay in order to treat and/or observe them longer. Such measures might decrease the risk of a short-term readmission; however, they increase the overall hospital costs. The longer the patient stays in the hospital, the higher the actual costs are and at some point, the costs of keeping a patient can reach and exceed the reimbursable flat-rate costs (“crossover point”). Moreover, as the number of hospital beds is a limited and oftentimes a scarce resource, extending occupancy by a patient inevitably means that the bed is not available for other patients.

### SUMMARY

By way of introduction, the preferred embodiments described below include embodiments for individual readmission risk prediction and discharge planning.

In a first aspect, a method is provided for individual readmission risk prediction, the method comprising: acquiring data about a patient; computing, using a time-varying readmission risk prediction model, a time-varying readmission risk prediction for the patient; presenting the time-varying readmission risk prediction in relation to a length of stay and a cost analysis; generating a discharge plan based on the presented time-varying readmission risk prediction and a pre-defined acceptable readmission risk threshold; and discharging the patient after the time-varying readmission risk prediction drops below the pre-defined acceptable readmission risk threshold.

In a second aspect, a system is provided for individual readmission risk prediction. The system includes a patient datastore, a hospital datastore, a time-varying readmission risk prediction model, and a discharge planner. The patient datastore is configured to store at least patient data. The hospital datastore is configured to store at least cost data for treatment of a patient and a reimbursement policy. The time-varying readmission risk prediction model is configured to generate a predicted readmission risk based on the patient data. The discharge planner is configured to generate a discharge plan based on the predicted readmission risk, the cost data, and the reimbursement policy.

In a third aspect, a non-transitory computer implemented storage medium is provided including machine-readable instructions stored therein, that when executed by at least one processor, cause the processor to: acquire data about a patient; compute, using a time-varying readmission risk prediction model, a time-varying readmission risk prediction for the patient; present the time-varying readmission risk prediction in relation to a length of stay and a cost analysis; generate a discharge plan based on the presented time-varying readmission risk prediction and a pre-defined acceptable readmission risk threshold; and generate instructions to discharge the patient after the time-varying readmission risk prediction drops below the pre-defined acceptable readmission risk threshold.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The components and the figures are not necessarily to scale, emphasis instead being placed upon illustrating the principles of the invention. Moreover, in the figures, like reference numerals designate corresponding parts throughout the different views.

FIG. 1 depicts a graph of actual costs and cost coverage.

FIG. 2 depicts a workflow for adaptive hospital discharge planning according to an embodiment.

FIG. 3 depicts a graph of a predicted readmission risk and costs savings.

FIG. 4 depicts a graph of a predicted readmission risk and costs savings.

FIG. 5 depicts a graph of a predicted readmission risk and additional costs.

FIG. 6 depicts a system for adaptive hospital discharge planning according to an embodiment.

### DETAILED DESCRIPTION

Embodiments provide systems and methods for reliable individual readmission risk prediction. Information about an admitted patient is acquired. A model inputs the patient information and outputs a time-varying readmission risk prediction. The time-varying readmission risk prediction is presented in a relation to the Length of Stay (LoS) and the costs (actual costs and cost coverage). A treatment and/or discharge plan is generated that is implemented when a threshold risk is crossed.

In an embodiment, the risk prediction is time-dependent and covers multiple time-points. Different models or analysis may be used to generate the time-varying readmission risk prediction. For example, the model may implement a regularized Cox proportional hazard model, a random survival forest, joint modeling, multi-task logistic regression, or others. The model is configured to estimate time-varying individual readmission risk based on available demographic data (e.g., age, gender), clinical data (e.g., laboratory blood values), examination data (e.g., blood pressure), medication data (e.g., drugs and dosages) and other data (e.g., number of previous admissions in the last 6 months, county etc.) data.

The systems and methods are applied dynamically. Embodiments recompute and adapt their predictions whenever new data becomes available (e.g., new patient examination that produces new data). When implemented, treating clinicians (or the hospital) may define an acceptable readmission risk threshold and plan to discharge the patient once the predicted individual readmission risk drops below the threshold. If the threshold cannot be reached before an upper length of stay, the patient may not be discharged. Otherwise, an acute readmission might happen that is not reimbursable, or the hospital might need to pay the penalties. On the other hand, as soon as the predicted risk reaches the threshold, the hospital might consider discharging the patient. Discharge of the patient, if done at the right time, might bring significant cost savings and free up resources (beds, staff, etc.). The dynamic nature of the prediction models allows the hospital to adapt to make better and more efficient discharge plans and decisions.

In addition to the passive use of the systems and methods for discharge management, embodiments may also be used actively as well. Instead of or in addition to computing the readmission risk for already decided treatment measures, in an embodiment, the risk prediction is simulated for various available treatment measures or options for the patient. In this case, the models may input the treatment options in addition to the length of stay, costs, and other relevant patient data. The model and resulting predicted readmission risks may be used to select a measure that minimizes the length of stay necessary to reach an acceptable level of readmission risk.

As described above, there are many factors associated with readmissions. Some are intrinsic, attributable to the reduced patient reserve due to disease progression and severity at each admission. Some may relate to the clinical planning and care coordination while patients are still in the hospital. Others may relate to post discharge care and other social factors. Current readmission risk adjustment models mostly rely on single point computations and/or standardized charts, for example a readmission risk calculated at the time of discharge. This poses an issue for patients, clinicians, and the hospital. Patients might not had been treated adequately or stabilized enough before they were discharged. Furthermore, readmissions also pose burden for their social circles e.g., their family members and friends. With a readmission, clinicians must deal with both newly admitted and readmitted patients and whose competence might be questioned by readmitted patients. Finally, as described above, the Hospital loses capacity and revenue. The hospital might have to pay penalties to payers or cannot request another, second flat-rate payment from payers if patients get readmitted within a certain timeframe after discharge.

There are at least three problems with existing readmission policies. A first problem is a lack of transparency regarding the trade-off between the individual patient readmission risk and the necessary length of stay (which correlates positively with the costs). A second related problem is the lack of methods to measure when it is safe to discharge the patient without risking too much, for example that the patients get readmitted within the upper length of stay. As described above, in such cases in some countries the hospitals cannot reimburse their actual readmission costs. A third problem is the lack of quantifiable impact of various treatment measures available to the clinician. If a function describing the relation between various treatments and the length of stay (or readmission risk) might be estimated with sufficient accuracy, it might be used for simulation and treatment optimization. Reliable individual readmission risk prediction is the most important feature necessary for addressing all three abovementioned problems.

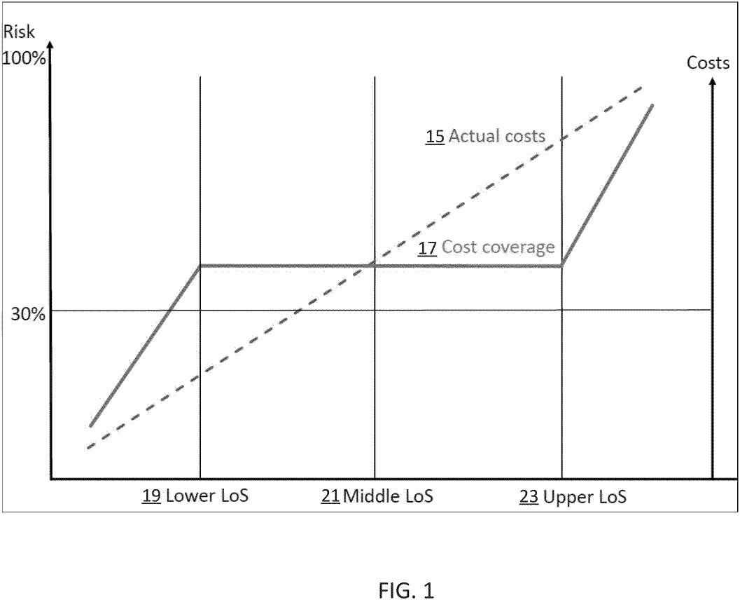

An example scenario regarding the costs of readmission is depicted in FIG. 1. The red dashed line shows the actual costs 15 which grow with a length of stay. The green line shows the flat-rate reimbursable costs 17 (cost coverage). There are three points in time designated by the Lower LoS 19, Middle LoS 21, and Upper LoS 23 that correspond to a reimbursement policy. A hospital may be reimbursed at a certain rate up to the Lower LoS 19 at which point no more reimbursement occurs. The Hospital is incentivized to treat the patient during this time. At some point, the actual costs 15 reach and cross over the possible cost coverage 17, for example at the point of the middle length of stay (Middle LoS 21). Ideally, the hospital would stabilize the patient sufficiently to avoid short-term readmission and discharge them before this happens. Otherwise, the patient would be discharged too early and potentially readmitted for the same reason. For example, if a readmission happens within a certain time frame (prior to the upper length of stay 23, defined by corresponding regulation in some countries such as Germany), the readmission case is treated as the original case and hospital is not able to reimburse a second flat-rate cost for this patient. For example, if the upper length of stay 23 is defined as twenty days, the patient gets discharged on day fifteen after the admission and gets readmitted on day nineteen (< upper length of stay 23), the original admission and readmission are considered to be the same case and no second flat-rate reimbursement is possible for the hospital. However, if the patient gets readmitted on the day twenty-three after the admission (> upper length of stay 23), the original admission and readmission are treated as separate cases, and each may be reimbursed separately.

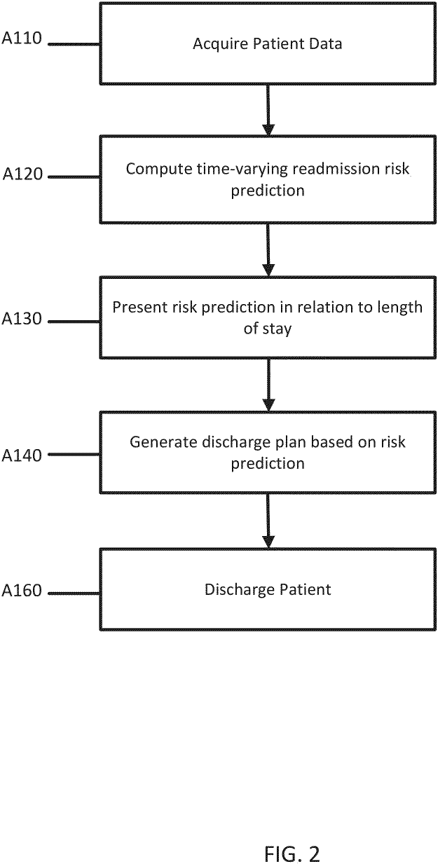

FIG. 2 depicts an example workflow for predicting individual readmission risk. The individual readmission risk model is configured to output a predicted readmission risk for a patient. The predicted readmission risk is input to the discharge component that is configured to generate and execute a discharge plan. The follow component acquires follow up data on the patient to be used as feedback / training data for the individual readmission risk model.

At act A110, the server acquires data about a patient. The data may be any data that is found to be relevant to readmission. In an embodiment, the data includes demographic data, lab data, examination data, imaging data, etc. The data about the patient may be stored in a patient datastore, for example an electronic medical record or chart. The patient data may include demographic data such as age, sex, medical history, weight, family history, diet, county, etc. The patient data may include image data, from for example, medical imaging devices. The patient data may also include lab test result such as (a) serum chemistry: albumin, aspartate transaminase, alkaline phosphatase, blood urea nitrogen, calcium, creatinine, glucose, potassium, sodium, and total bilirubin; (b) hematology and coagulation parameters: bands, hemoglobin, partial thromboplastin time, prothrombin time international normalized ratio, platelets, and white blood cell count; (c) arterial blood gas: partial pressure of carbon dioxide, partial pressure of oxygen, and pH value; (d) cardiac markers: brain natriuretic peptide, creatine phosphokinase MB, probrain natriuretic peptide, and troponin I, etc. The patient data may be annotated by a clinician or other medical professional.

Different sets of data may be used in training or for configuring the individual readmission risk model. As an example, certain characteristics such as age, sex, payer, illness severity, whether admitted through the emergency department, and patient residence in the same county as the hospital may be significantly associated with readmissions. The patient data may be limited to only those characteristics upon which readmission is dependent from. Different diagnosis may use different readmission risk models that are configured to input different types or sets of data. For example, a HF patient’s readmission risk may be dependent on specific characteristics that are particular to HF patients.

In an embodiment, patient data is collected during at multiple points during the entirety of the patient’s stay in the hospital until discharged. The Individual Readmission Risk Model as described below may adapt its predictions based on newly acquired or collected data. Many known readmission prediction models are only implemented at the time of patient discharge. However, interventions that include an early in-hospital component are critical in reducing readmissions and improving patient outcomes. Thus, at-discharge high-risk identification may be too late for effective intervention (and does not provide any guidance in when to discharge). Patient data may also be collected about a time period prior to admission and/or after discharge. Patient data related to a time period after readmission may be used as feedback for the Individual Readmission Risk Model and/or for discerning beneficial outcomes of treatment options.

At act A120, the Individual Readmission Risk Model computes a time-varying readmission risk prediction for the patient in relation to a length of stay. A readmission risk for a patient may drop over time as the patient receives additional care. As an example, the Individual Readmission Risk Model may predict a much higher chance of readmission if a patient is discharged on day 4 instead of day 10. The readmission risk prediction may be recomputed daily or as new information about the patient becomes available (for example how the patient responds to certain medications or how the values for lab tests are diagnosed). The readmission risk may be computed at regular intervals or upon a request by a clinician. Different risk models may be implemented for different patient ailments. In an embodiment, the risk model may be configured or trained using ground truth data at any point prior to implementation. For example, the risk model may be trained using a set of data for HF patients that are annotated with when or if the patient was readmitted.

The Individual Readmission Risk Model may implement a survival analysis to predict the occurrence of readmission. Survival analysis may be used to analyze or predict when an event such as readmission is likely to happen. The survival models include two parts: an underlying baseline hazard function describing how the risk of event per time unit changes over time at baseline levels of covariates; and the effect parameters, describing how the hazard varies in response to explanatory covariates. A typical medical example would include covariates such as the diagnosis, as well as the patient characteristics. Survival analysis is used to predict the timing of an event of interest, such as the readmission a patient. Survival methods handle the common situation of right censoring that arises when a patient is readmitted after the window of observation due to dropout or study termination (other types of censoring are possible). The endpoint or outcome of survival data includes the two random variables of the time to the event and a censoring code (0 if censored, 1 if event).

In an embodiment, the Individual Readmission Risk Model uses a proportional hazards model. Proportional hazards models are a class of survival models. The proportional hazards model relates the time that passes, before readmission occurs, to one or more covariates that may be associated with that quantity of time. Using a proportional hazards model, the unique effect of a unit increase in a covariate is multiplicative with respect to the hazard rate. For example, discharging a patient one day later may halve the hazard rate for readmission occurring. The Cox Proportional Hazard model (CoxPH) is a type of a proportional hazards model that is semi-parametric and focuses on modeling the hazard function, by assuming that its time component and feature component are proportional. In addition to allowing time-varying covariates (i.e., predictors), the Cox model may be generalized to time-varying coefficients as well. That is, the proportional effect of a treatment may vary with time; e.g., a discharge may be less effective if effected within a few days of admittance and become more effective as time goes on. The hypothesis of no change with time (stationarity) of the coefficient may then be tested.

In an embodiment, the Individual Readmission Risk Model uses a Multi-Task Logistic Regression model. The Multi-Task Logistic Regression (MTLR) model is a series of logistic regression models built on different time intervals to estimate the probability that the event of interest happened within each interval. Although the MTLR model provides similar results as the CoxPH model without having to rely on the assumptions required by the latter, it is still powered by a linear transformation. Thus, in the presence of nonlinear elements in the data, it will stop yielding satisfactory performances. A Neural Multi-Task Logistic Regression (N-MTLR) that uses neural networks within the MTLR framework may solve this issue.

In an embodiment, the Individual Readmission Risk Model uses a random survival forest model or RSF. Models that use decision trees as its base learners may be generally grouped under the name Survival Forest models. A RSF is an extension of the Random Forest model that is able to use censoring. The RSF is an assemble of trees method for analysis of right censored time-to-event data. Survival trees and forests are popular non-parametric alternatives to (semi) parametric models for time-to-event analysis. A survival tree is built with the idea of partitioning the covariate space recursively to form groups of subjects who are similar according to the time-to-event outcome. Homogeneity at a node is achieved by minimizing a given impurity measure. The basic approach for building a survival tree is by using a binary split on a single predictor. The goal in survival tree building is to identify prognostic factors that are predictive of the time-to-event outcome, e.g., readmission of the patient.

The underlying algorithm that powers the Survival Forest model includes the following steps. First, the model draws random samples of the same size from an original dataset with replacement. The samples that are not drawn are said to be out-of-bag (OOB). A survival tree is grown for each of the samples. At each node of the tree, a random subset of predictor variables is selected and the best predictor and splitting value are identified that provide two subsets (the daughter nodes) that maximizes the difference in the objective function. This is repeated recursively on each daughter node until a stopping criterion is met. A cumulative hazard function (CHF) is calculated for each tree and averaged over all CHFs for the B trees to obtain the ensemble CHF. The prediction error for the ensemble CHF is computed using only the OOB data.

In an embodiment, the Individual Readmission Risk Model uses Joint modeling. Joint modeling (JM) is appropriate when one wants to predict the time to an event with covariates that are measured longitudinally and are related to the event. An underlying random effect structure links the survival and longitudinal sub models and allows for individual-specific predictions. Multiple time-varying and time-invariant covariates can be included to potentially increase prediction accuracy. The JM survival model may take several forms, with the proportional hazards model being a common choice. In this scenario, the goal of JM is to predict the hazard (or the log hazard) of readmission. Covariates can be time-varying (i.e., longitudinal) or time-invariant. A time-invariant covariate is directly specified in the survival sub model like the traditional model. Each time-invariant covariate has a corresponding regression coefficient that indicates the strength of the covariate in predicting the hazard, adjusting for the other covariates. Rather than include a time-varying covariate directly in the survival sub model, the longitudinal information is specified through a function of a separate but interdependent longitudinal sub model for the covariate. A popular choice of function is the underlying or “true” value of the covariate that occurs contemporaneously with the hazard. Each true value predictor has a regression coefficient indicating its effect on the hazard.

The JM longitudinal sub model is a linear mixed model (LMM) for a continuous covariate, or a generalized LMM for a discrete covariate (e.g., binary variable). A time-varying covariate is the outcome in the LMM, but the true value of the covariate is a predictor in the JM survival sub model. It is in this manner that the two sub models are linked, with random effects being shared among the sub models. When there is more than one time-varying covariate, then the LMM is referred to be multivariate. Multivariate refers to multiple interdependent LMMs with different outcomes (the predictors in each LMM might or might not be the same). The different LMMs are interdependent through a shared random effects structure. In the JM survival submodel each true covariate is a predictor and has an associated regression coefficient indicating its strength of relationship with the hazard, adjusting for the other predictors in the model (whether they be other true covariate values or time-invariant covariates).

The risk model may include or implement a neural network, for example when using N-MTLR or JM. The model may be trained using machine learning techniques. The model may be trained using supervised or unsupervised learning. The model(s) may include a neural network that is defined as a plurality of sequential feature units or layers. Sequential is used to indicate the general flow of output feature values from one layer to input to a next layer. Sequential is used to indicate the general flow of output feature values from one layer to input to a next layer. The information from the next layer is fed to a next layer, and so on until the final output. The layers may only feed forward or may be bi-directional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction based on the input data. The features are learned to reconstruct lower-level features (i.e., features at a more abstract or compressed level). Each node of the unit represents a feature. Different units are provided for learning different features. Various units or layers may be used, such as convolutional, pooling (e.g., max pooling), deconvolutional, fully connected, or other types of layers. Within a unit or layer, any number of nodes is provided. For example, 100 nodes are provided. Later or subsequent units may have more, fewer, or the same number of nodes. Unsupervised learning may also be used based on the distribution of the samples, using methods such as k-nearest neighbor.

Different neural network configurations and workflows may be used for or in the model such as a convolution neural network (CNN), deep belief nets (DBN), or other deep networks. CNN learns feed-forward mapping functions while DBN learns a generative model of data. In addition, CNN uses shared weights for all local regions while DBN is a fully connected network (e.g., including different weights for all regions of a feature map. The training of CNN is entirely discriminative through backpropagation. DBN, on the other hand, employs the layer-wise unsupervised training (e.g., pre-training) followed by the discriminative refinement with backpropagation if necessary. In an embodiment, the arrangement of the trained network is a fully convolutional network (FCN). Other network arrangements may be used, for example, a 3D Very Deep Convolutional Networks (3D-VGGNet). VGGNet stacks many layer blocks containing narrow convolutional layers followed by max pooling layers. A 3D Deep Residual Networks (3D-ResNet) architecture may be used. A Resnet uses residual blocks and skip connections to learn residual mapping.

The training data for the model (and other networks) includes ground truth data or gold standard data, for example datasets collected over a period that include both patient data and an indication of whether a readmission occurred. Ground truth data and gold standard data is data that includes correct or reasonably accurate labels that are verified manually or by some other accurate method. The training data may be acquired at any point prior to inputting the training data into the model. The model may input the training data (e.g., the patient data) and output a time varying prediction for readmission. The prediction is compared to the annotations from the training data (for example, if there was a readmission event or not). A loss function may be used to identify the errors from the comparison. The loss function serves as a measurement of how far the current set of predictions are from the corresponding true values. Some examples of loss functions that may be used include Mean-Squared-Error, Root-Mean-Squared-Error, and Cross-entropy loss. Mean Squared Error loss, or MSE for short, is calculated as the average of the squared differences between the predicted and actual values. Root-Mean Squared Error is similarly calculated as the average of the root squared differences between the predicted and actual values. For cross-entropy loss each predicted probability is compared to the actual class output value (0 or 1) and a score is calculated that penalizes the probability based on the distance from the expected value. The penalty may be logarithmic, offering a small score for small differences (0.1 or 0.2) and enormous score for a large difference (0.9 or 1.0). During training and over repeated iterations, the network attempts to minimize the loss function as the result of a lower error between the actual and the predicted values means the network has done a good job in learning. Different optimization algorithms may be used to minimize the loss function, such as, for example, gradient descent, Stochastic gradient descent, Batch gradient descent, Mini-Batch gradient descent, among others. The process of inputting, outputting, comparing, and adjusting is repeated for a predetermined number of iterations with the goal of minimizing the loss function. Once adjusted and trained, the model is configured to generate a time-varying readmission risk prediction in real time.

At act A130, the model outputs the time-varying readmission risk prediction in relation to a length of stay. The time-varying readmission risk prediction may predict different readmission risks as the length of stay is cut short or extended. For example, the model may predict a high chance of readmission if the patient is discharge early eventually dropping as the length of stay is extended. As described above and below, the model may recompute or adapt the risk prediction as new information is received.

At act A140, the system generates a discharge plan based on the time-varying readmission risk prediction and a pre-defined acceptable readmission risk threshold. The pre-defined acceptable readmission risk threshold may be defined by a hospital or clinician. The pre-defined acceptable readmission risk threshold may depend on a regulation or reimbursement policy. Discharge planning may be effective at reducing hospital readmissions. A comprehensive discharge plan must consider the entire scope of a patient’s health needs and should include input and feedback from both the patient and family. The discharge plans may include written, visual, or recorded information including follow-up appointments, medications, nutritional needs, family and patient support, transportation, health literacy, resources to call, social problems, and red flags. The discharge plan may provide for a discharge in the future, for example in one, two, five, etc. days from when the discharge plan is created. As an example, the risk model may predict that after receiving treatment for two additional days, a patient’s readmission prediction may drop below an acceptable threshold and therefore the patient will be eligible for discharge. The discharge plan may be recomputed or adapted over time as the patient responds to treatments. By using a time varying prediction, a discharge plan may be generated in advance. This allows a hospital to plan ahead for capacity, staffing, and other needs. The adaptive discharge plan further allows for efficient care and support for the patient by providing answers ahead of time during a stay that can be stressful to the patient and their support network.

In an embodiment, the discharge plan considers costs, for example active costs and reimbursement policies for the hospital. Active costs may include the actual costs 15 for treating a patient. Actual costs 15 increase over time, for example linearly, as each day spent at the hospital incurs some cost to the hospital. Treatment or testing costs may also be included in the active costs. Reimbursement policies or cost coverage 17 may be country or location specific depending on regulations or contracts. Reimbursement costs may be represented by a step function or a non-linear function. Reimbursement costs generally increase over time but may have limits or boundaries. For example, due to understandings with insurance companies or governmental regulations, reimbursement policies may provide where reimbursement is capped for certain patients, treatments, diagnosis, etc. The discharge plan considers these financial aspects in relation to the length of stay (LoS). While the primary goal is to care for patients, a hospital that operates financially responsible is able to provide better and efficient care for more patients and thus provides a benefit to society as whole.

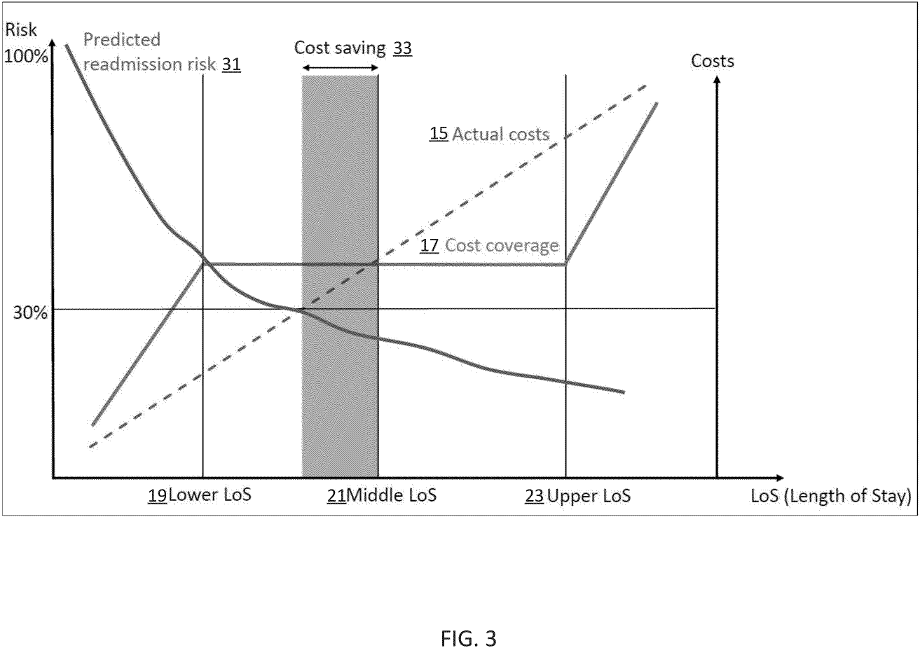

FIG. 3 depicts an example time-varying readmission risk prediction. In FIG. 3, the predicted readmission risk 31 diminishes over time as the length of stay increases. The pre-defined acceptable readmission risk threshold is set at 30% here. FIG. 3 further includes the actual costs 15 and cost coverage 17 from FIG. 1. As depicted, the predicted readmission risk 31 falls below the threshold prior to the Middle LoS 21 (or cross over point). If the patient is discharged at this point, the reimbursement policy will cover the actual costs 15 while limiting the readmission risk. This provides costs savings 33 to the hospital. The predicted readmission risk 31 may be recalculated during the patient’s stay in order to more accurate predict the risk as the point of discharge comes closer.

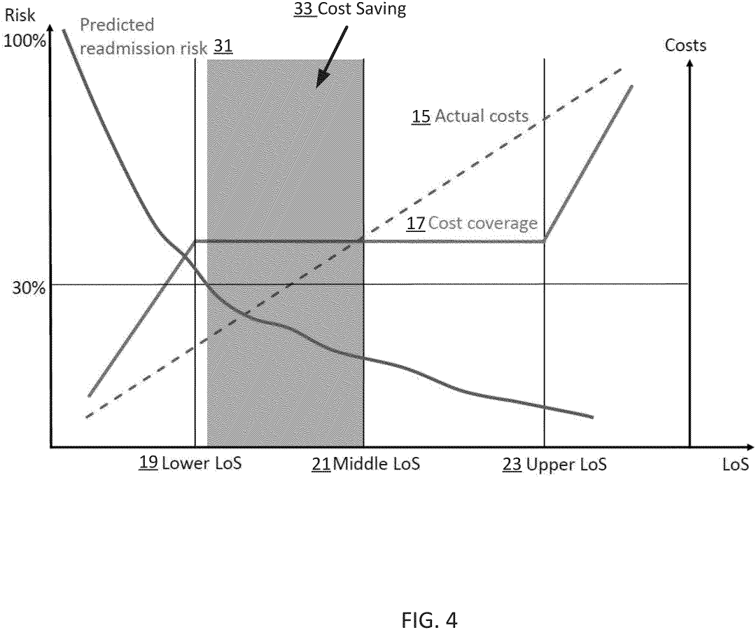

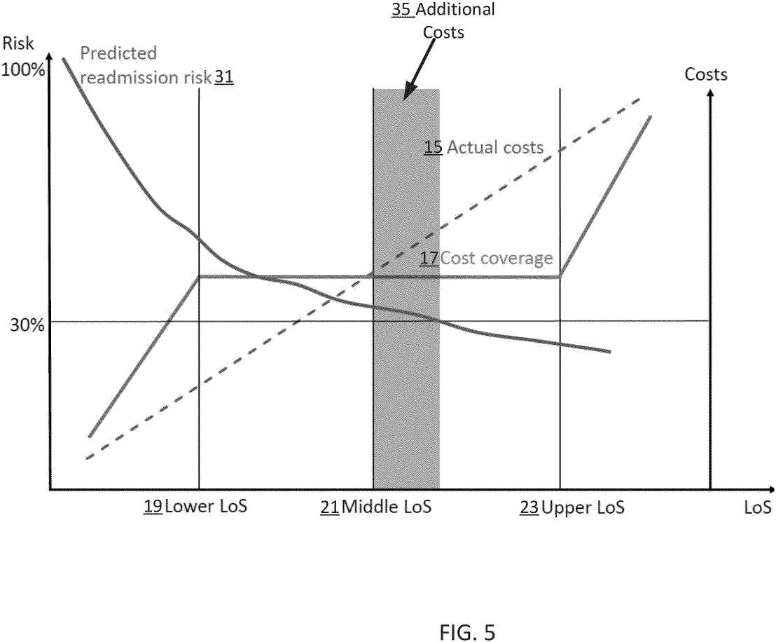

FIGS. 4 and 5 depict different examples for which to base the discharge plan on. In FIG. 4, the predicted readmission risk 31 drops below threshold of 30% much sooner than in FIG. 3. If the prediction stays the same (the model may provide new predictions as new data is acquired) and the discharge is planned and executed, the hospital and patient will both benefit from a shorter stay (e.g., costs savings 33 for the hospital). In FIG. 5, the predicted readmission risk 31 does not drop below the threshold of 30% until after the cross-over point. This may indicate that in order to provide acceptable care, the hospital may need to suffer a loss (Additional costs 35) as the actual costs 15 exceed the cost coverage 17. Alternatively, such a predicted readmission risk 31, if identified early enough in the patient’s stay may allow a clinician to alter a treatment of the patient in order to attempt to lower the risk prior to the cross over point.

In an embodiment, in addition to the passive use of the proposed framework for discharge management, the time-varying readmission risk prediction may be used actively as well. Instead of or in addition to computing the readmission risk for already decided treatment measures, a clinician may simulate the risk prediction for various available measures, for example to lower the predicted readmission risk 31. In this case, the time-varying readmission risk prediction model may take the treatment options in addition to the LoS and all other relevant patient data as inputs. The simulation may be used to select the measure M that minimizes the length of stay necessary to reach an acceptable level of readmission risk (e.g., 30% as predefined by clinicians and hospital management). For example, the predicted readmission risk 31 curve of FIG. 4 is preferable to the predicted readmission risk 31 curve of FIG. 5. A clinician may use the model to identify which treatments generate the curve of FIG. 4 or at least reach a discharge point prior to the cross-over point. A clinician / hospital may have several options for how they deal with different treatments, thresholds, and costs. The result is an optimized discharge plan in light of the possible treatments for the patient.

As an example, there may be four different scenarios that can be used to compute the discharge plan. For a maximized ratio the cost coverage 17 / actual cost for LoS <= Middle LoS 21 to maximize savings. For a minimized ratio, the actual cost / cost coverage 17 for Middle LoS 21 <= LoS <= Upper LoS 23 to minimize loss. For a minimized LoS, the discharge plan focuses on when the acceptable readmission risk is reached (i.e., minimizing the risk that hospital will have to pay penalties for readmission). For a minimized LoS in all cases which frees hospital beds and other resources and potentially lowers the work burden for the hospital stuff. Using the readmission prediction, the discharge plan may thus be set to provide maximum savings, minimize loss, maximize patient health, or maximize hospital operation. A combination of these goals may also be implemented, for example attempting to maximize savings while providing an acceptable level of patient health and while allowing some breathing room for staffing and operation of the hospital.

At act A150, the hospital discharges the patient after the predicted individual readmission risk drops below the threshold as, for example, specified in the discharge plan. The time-varying readmission risk prediction may provide a reduction in hospital readmissions and thus have potential financial benefits in the form of lower penalties. A decrease in the number of readmissions may have broader benefits, for example, it might increase hospitals’ ability to manage patient resources and increase their ability to manage post-acute care, resulting in improved community health and higher patient satisfaction. Patients may also benefit by being provided a discharge plan earlier in the process. An adaptive plan also allows a patient to understand how their treatment is progressing. Finally, using a predefined threshold may minimize the cost to a patient by preventing unnecessary longer stays while providing quality care.

The workflow of FIG. 2 may be performed by a discharge planner software or hardware system. The discharge planner coordinates patient information and care into a single interface that may be accessed by a clinician or a patient. When provided patient information, the discharge planner may provide an estimated discharge date and treatment options. A hospital may configure the discharge planner with one or more settings that operate in conjunction with the time-varying readmission risk prediction model. For example, the hospital may set a threshold risk, set costs, set the reimbursement levels etc. The discharge planner predicts when these conditions or settings are met considering the time varying prediction by the time-varying readmission risk prediction model. An estimated length of stay may thus be predicted for the patient at various points in time from admission through discharge. In addition, the discharge planner may allow a clinician the ability to model various treatments in order to lower the risk to a patient.

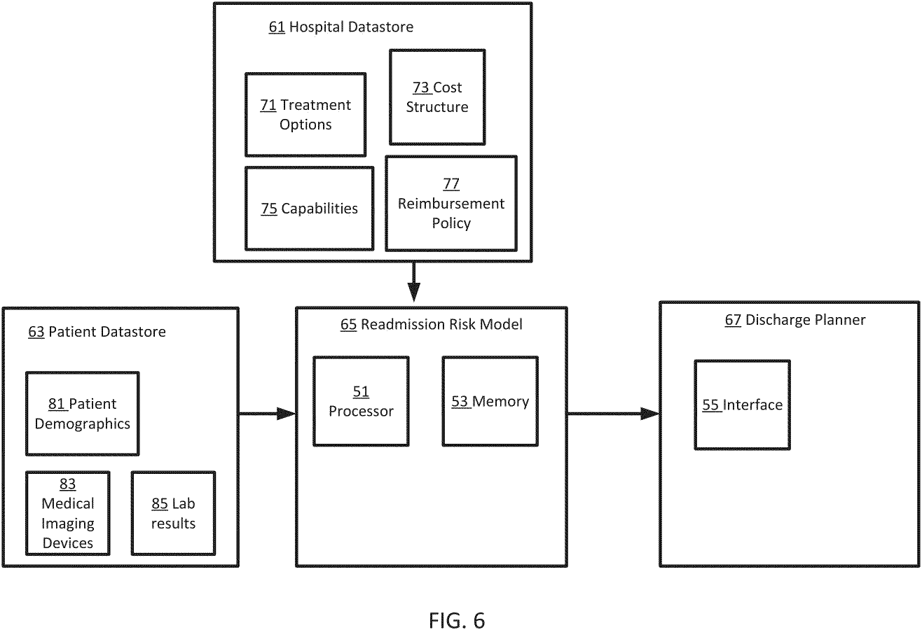

FIG. 6 depicts an example discharge planning system for predicting individual readmission risk. The system includes a patient datastore 63, a hospital datastore 61, a time-varying readmission risk model 65, and a discharge planner 67. Additional, different, or fewer components may be provided. For example, network connections or interfaces may be provided, such as for networking with a medical imaging network or data archival system. In an embodiment, the time-varying readmission risk model 65 inputs data from the patient datastore 63 and the hospital datastore 61. The time-varying readmission risk model 65 provides a predicted risk to a discharge planner that generates a discharge plan.

The time-varying readmission risk model 65 includes at least a memory 53 and a processor 51. The time-varying readmission risk model 65 may share an interface 55 with the discharge planner 67. The memory 53 may be a graphics processing memory, a video random access memory, a random-access memory, system memory, cache memory, hard drive, optical media, magnetic media, flash drive, buffer, database, combinations thereof, or other now known or later developed memory device for storing data or video information. The time-varying readmission risk prediction model 65 may be part of a control unit, server, part of a computer associated with the hospital, part of a database, part of another system, or a standalone device.

The memory 53 may be a non-transitory computer readable storage medium storing data representing instructions executable by the processor for time-varying readmission risk prediction. The instructions for implementing the processes, methods and/or techniques discussed herein are provided on non-transitory computer-readable storage media or memories, such as a cache, buffer, RAM, removable media, hard drive, or other computer readable storage media. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, processor or processing strategy and may be performed by software, hardware, integrated circuits, firmware, micro code, and the like, operating alone, or in combination. Likewise, processing strategies may include multiprocessing, multitasking, parallel processing, and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system. The memory 53 may store a model or machine learnt network.

The processor 51 is a general processor, central processing unit, control processor, graphics processing unit, digital signal processor, three-dimensional rendering processor, image processor, application specific integrated circuit, field programmable gate array, digital circuit, analog circuit, combinations thereof, or other now known or later developed device for processing medical imaging data. The processor 51 is a single device or multiple devices operating in serial, parallel, or separately. The processor 51 may be a main processor of a computer, such as a laptop or desktop computer, or may be a processor for handling some tasks in a larger system, such as in the server. The processor 51 is configured by instructions, design, hardware, and/or software to perform the acts discussed herein.

The processor 51 is configured to implement the time-varying readmission risk prediction model 65 by inputting various forms of data from the patient datastore 63 and/or hospital datastore 61. The processor 51 is also configured to train, configure, adapt, and/or maintain the time-varying readmission risk prediction model 65, using for example machine learning techniques. The processor 51 may acquire one or more training sets of data relating to whether a patient was readmitted or not. The training data may be used to configure the time-varying readmission risk prediction model 65 to provide more accurate predictions of the risk of readmission. The time-varying readmission risk prediction model 65 may be updated or adapted by the processor 51 as additional data is collected.

The patient datastore 63 may acquire and/or store data relating to a patient such as patient demographics 81, medical imaging device data 83, or lab results 85. The patient datastore 63 may include, for example, a digitalized health record or chart. The medical chart may be a complete record of a patient’s key clinical data and medical history, such as demographics, vital signs, diagnoses, medications, treatment plans, progress notes, problems, immunization dates, allergies, radiology images, and laboratory and test results. Sample data may include surgical history (e.g., operation dates, operation reports, operation narratives), obstetric history: (e.g., pregnancies, any complications, pregnancy outcomes), medications and medical allergies, family history (e.g., immediate family member health status, cause of death, common family diseases), social history (e.g., community support, close relationships, past and current occupation), Habits (e.g., smoking, alcohol consumption, exercise, diet, sexual history), Immunization Records (e.g., vaccinations, immunoglobulin test), Developmental History (e.g., growth chart, motor development, cognitive/intellectual development, social-emotional development, language development), Demographics (e.g., race, age, religion, occupation, contact information), and medical encounters (e.g., hospital admissions, specialist consultations, routine checkups) among other data. The medical chart may also include medical notes made by a physician, nurse, lab technician or any other member of a patient’s healthcare team. This data may include, for example, chief complaints, a history of the present illness, physical examination (e.g., vital signs, muscle power, organ system examinations), assessment and plan (e.g., diagnosis, treatment), orders and prescriptions, progress notes, and test results (e.g., imaging results, pathology results, specialized testing).

The hospital datastore 61 may store data that is related to the hospital, for example, the capabilities (staffing, equipment), the cost structure, reimbursement policies, and possible treatments. Different hospitals may have different characteristics and capabilities. The time-varying readmission risk prediction model 65 may be configured for an individual hospital based on the hospital data or, for example, may be a universal model that can be used across an entire system.

The discharge planner 67 may be configured to receive prediction data from the readmission risk model 65, the patient datastore 63, and the hospital datastore 61. The discharge planner 67 is configured to plan a discharge of a patient based on a readmission risk prediction, costs, hospital capabilities, etc. The discharge planner 67 may include an interface 55 for a clinician to interact with. The interface 55 may be a monitor, LCD, projector, plasma display, CRT, printer, or other now known or later developed devise for outputting visual information. The interface receives images, graphics, text, quantities, or other information from the processor, memory, and/or server. The interface is configured to provide images and other data related to a readmission risk / discharge plan to an operator. The interface may include an input device such as one or more buttons, a keypad, a keyboard, a mouse, a stylus pen, a trackball, a rocker switch, a touch pad, a voice recognition circuit, or other device or component for inputting data. The interface and a display may be combined as a touch screen that may be capacitive or resistive. The interface may further interact with the individual readmission risk model 65 to adjust settings, preferences, thresholds, etc. The interface may interact with the hospital data to adjust or set the cost structure / reimbursement structure, select treatment options, or adjust capabilities such as staffing or equipment availability.

The patient datastore 63, hospital datastore 61, risk model 65, and discharge planner 67 may be connected using a network. The network is a local area, wide area, enterprise, another network, or combinations thereof. In one embodiment, the network is, at least in part, the Internet. Using TCP/IP communications, the network provides for communication between the processor and the server. Any format for communications may be used. In other embodiments, dedicated or direct communication is used.

While the invention has been described above by reference to various embodiments, many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for individual readmission risk prediction, the method comprising: acquiring data about a patient;
computing, using a time-varying readmission risk prediction model, a time-varying readmission risk prediction for the patient;
presenting the time-varying readmission risk prediction in relation to a length of stay and a cost analysis;
generating a discharge plan based on the presented time-varying readmission risk prediction and a pre-defined acceptable readmission risk threshold; and
discharging the patient after the time-varying readmission risk prediction drops below the pre-defined acceptable readmission risk threshold.

2. The method of claim 1, wherein the time-varying readmission risk prediction model comprises a Cox proportional hazard model.

3. The method of claim 1, wherein the time-varying readmission risk prediction model comprises a random survival forest model.

4. The method of claim 1, wherein the time-varying readmission risk prediction model comprises a multi-task logistic regression model.

5. The method of claim 1, wherein computing is repeated when new patient data becomes available.

6. The method of claim 1, further comprising:
selecting one or more treatments for the patient;
wherein the time-varying readmission risk prediction is computed for different treatments of the one or more treatments to estimate an optimal discharge plan.

7. The method of claim 1, wherein computing, presenting, and generating are performed at regular intervals or upon a clinician’s request during the patient’s stay.

8. The method of claim 1, wherein the cost analysis takes into account actual costs for the length of stay and a reimbursement policy.

9. The method of claim 8, wherein the reimbursement policy penalizes readmissions within a time period of admission.

10. A system for individual readmission risk prediction, the system comprising:
a patient datastore configured to store at least patient data;
a hospital datastore configured to store at least cost data for treatment of a patient and a reimbursement policy;
a time-varying readmission risk prediction model configured to generate a predicted readmission risk based on the patient data;
a discharge planner configured to generate a discharge plan based on the predicted readmission risk, the cost data, and the reimbursement policy.

11. The system of claim 10, further comprising an interface configured to display the discharge plan.

12. The system of claim 10, wherein the time-varying readmission risk prediction model comprises a Cox proportional hazard model.

13. The system of claim 10, wherein the time-varying readmission risk prediction model comprises a random survival forest model.

14. The system of claim 10, wherein the time-varying readmission risk prediction model comprises a multi-task logistic regression model.

15. The system of claim 10, wherein the time-varying readmission risk prediction model is configured to adapt the predicted readmission risk when providing new patient data.

16. The system of claim 10, wherein the reimbursement policy penalizes readmissions within a time period of admission.

17. The system of claim 10, wherein the discharge planner is further configured to generate the discharge plan using a predefined threshold for the predicted readmission risk.

18. A non-transitory computer implemented storage medium, including machine-readable instructions stored therein, that when executed by at least one processor, cause the processor to:
acquire data about a patient;
compute, using a time-varying readmission risk prediction model, a time-varying readmission risk prediction for the patient;
present the time-varying readmission risk prediction in relation to a length of stay and a cost analysis;
generate a discharge plan based on the presented time-varying readmission risk prediction and a pre-defined acceptable readmission risk threshold; and
generate instructions to discharge the patient after the time-varying readmission risk prediction drops below the pre-defined acceptable readmission risk threshold.

19. The non-transitory computer implemented storage medium of claim 18, wherein the time-varying readmission risk prediction model comprises one of a Cox proportional hazard model, a random survival forest model, a multi-task logistic regression model, or any other model capable of computing the time-varying readmission risk prediction.

20. The non-transitory computer implemented storage medium of claim 18, wherein the cost analysis takes into account actual costs for the length of stay and a reimbursement policy.
